(19) United States
(12) Patent Application Publication
(10) Pub. No.: US 2024/0358851 A1
(43) Pub. Date: Oct. 31, 2024

(54) SHEAR-THINNING HYDROGELS AND USES THEREOF

(71) Applicant: The Research Foundation for the State University of New York, Amherst, NY (US)

(72) Inventors: Stelios Andreadis, East Amherst, NY (US); Mohamed Alaa Mohamed, Buffalo, NY (US)

(21) Appl. No.: 18/687,947

(22) PCT Filed: Aug. 30, 2022
(86) PCT No.: PCT/US2022/042036

§ 371 Date: Feb. 29, 2024

Related U.S. Application Data
(60) Provisional application No. 63/238,911, filed on Aug. 31, 2021.

(51) Int. Cl. A61K 47/69; A61K 38/18; A61K 45/06; A61K 47/36; A61K 47/60

(52) U.S. Cl. CPC A61K 47/6951; A61K 38/18; A61K 45/06; A61K 47/36; A61K 47/60

(57) ABSTRACT

The present disclosure is directed to shear-thinning and stabilizing hydrogels, especially for use in drug delivery, therapy, and 3D printing. Embodiments provide an injectable hydrogel, including: a water-soluble polymer including a first functional group; a host molecule including a second functional group, wherein the second functional group is characterized as complementary to the first functional group; and a guest-terminated star polymer, wherein the guest-terminated star polymer comprises a core unit, a plurality of arms extending from the core unit, and a guest molecule disposed at a terminal end of each of the plurality of arms, and wherein the host molecule and the guest-terminated star polymer are linked by one or more bonds that break under mechanical stress and reform after removal of the mechanical stress.

### CROSS REFERENCE TO RELATED APPLICATIONS

This application claims the benefit of the filing date of U.S. Provisional Application No. 63/238,911 filed Aug. 31, 2021. The content of this earlier filed application is hereby incorporated by reference herein in its entirety.

### GOVERNMENT SUPPORT

This invention was made with government support under grant no. EB023114 awarded by the National Institutes of Health. The U.S. Government has certain rights in the invention.

### FIELD OF THE INVENTION

The present disclosure is directed to hydrogel chemistry and/or host-guest polymer chemistry for providing injectable hydrogels such as shear-thinning supramolecular hydrogels, especially for use in drug delivery, cell delivery, therapy, and 3D printing.

### BACKGROUND

Hydrogels are three dimensional (3D) networks containing large quantities of water and may act as reservoirs for delivery of therapeutic agents, bioactive signals, and cells to diseased or injured sites in a subject in need thereof. Accordingly, hydrogels hold great promise in many applications such as drug delivery and tissue engineering.

Significant progress in tissue engineering has been achieved using biopolymer-based hydrogels such as fibrin-, collagen-, and gelatin-methacrylate-, dextran-, and polyvinyl alcohol-based hydrogels. However, the inventors have observed that known hydrogels problematically exhibit fast gelation kinetics, restricting or preventing hydrogel use in applications where they are delivered with shear force such as via injection. Furthermore, the inventors have observed that conventional hydrogels lack shear-thinning properties and therefore, cannot be used to deliver cells via injection as shear force leads to high cell death due to mechanical disruption of the cell membrane. Moreover, the inventors have observed that hydrogel derived from photo-reactive polymers often problematically require use of photo-initiators and cytotoxic doses of UV radiation.

Prior-art-of interest includes U.S. Pat. No. 9,827,321 (herein incorporated by reference) directed to shear-thinning and stabilizing hydrogels, for use in drug delivery and therapy. Further, U.S. Pat. No. 10,828,399 (herein incorporated by reference) is directed to methods of printing 3D structures using supramolecular gels, and the structures that result therefrom. However, the references are deficient and do not provide for shear-thinning hydrogels with controlled mechanical properties in accordance with the present disclosure.

There is a continuous need for new hydrogels with controlled mechanical properties suitable for use in, inter alia, cell delivery, treatments, and 3D printing. What is needed is supramolecular chemistry and/or guest/host polymer chemistry to alter or tune properties of hydrogels such as mechanical/rheological properties and/or dynamic properties such as kinetics of bond formation.

### SUMMARY

The present disclosure relates shear-thinning supramolecular hydrogels and/or shear-thinning hydrogels, especially for use in drug delivery, cell delivery, therapy, and 3D printing.

In embodiments, the present disclosure includes an injectable hydrogel, including: a water-soluble polymer including a first functional group; a host molecule including a second functional group, wherein the second functional group is characterized as complementary to the first functional group; and a guest-terminated star polymer, wherein the guest-terminated star polymer includes a core unit, a plurality of arms extending from the core unit, and a guest molecule disposed at a terminal end of each of the plurality of arms, and wherein the host molecule and the guest-terminated star polymer are linked by one or more bonds that break under mechanical stress and reform after removal of the mechanical stress.

In embodiments, the present disclosure relates to a shear-thinning supramolecular hydrogel, including: a water-soluble polymer including a first functional group; a host molecule including a second functional group, wherein the second functional group is characterized as complementary to the first functional group; and a guest-terminated star polymer, wherein the guest-terminated star polymer includes a core unit, a plurality of arms extending from the core unit, and a guest molecule disposed at a terminal end of each of the plurality of arms, and wherein the host molecule and the guest-terminated star polymer are linked by one or more bonds that break under mechanical stress and reform after removal of the mechanical stress.

In some embodiments, the present disclosure relates to a hydrogel, including: a water-soluble polymer including a first functional group; a host molecule including a second functional group, wherein the second functional group is characterized as complementary to the first functional group; and a guest-terminated star polymer, wherein the guest-terminated star polymer includes a core unit, a plurality of arms extending from the core unit, and a guest molecule disposed at a terminal end of each of the plurality of arms, and wherein the host molecule and the guest-terminated star polymer are linked by one or more bonds that break under mechanical stress and reform after removal of the mechanical stress, wherein the guest-terminated star polymer is an n-arm-PEG adamantane, wherein n is an integer characterized as 2, 4, 6, or 8.

In some embodiments, the present disclosure relates to a bioactive hydrogel, wherein the bioactive hydrogel is prepared by: contacting a water-soluble polymer including a first functional group, a host molecule including a second functional group, wherein the second functional group is characterized as complementary to the first functional group, and a guest-terminated star polymer, wherein the guest-terminated star polymer includes a core unit, a plurality of arms extending from the core unit, and a guest molecule disposed at a terminal end of each of the plurality of arms, and wherein the host molecule and the guest-terminated star polymer are linked by one or more bonds that break under mechanical stress and reform after removal of the mechanical stress to form a mixture, wherein the contacting is performed under conditions suitable for forming a hydrophilic polymer network from the mixture.

In some embodiments, the present disclosure relates to a method of making a hydrogel, including: contacting (1) a water-soluble polymer including a first functional group, (2) a host molecule including a second functional group, wherein the second functional group is characterized as complementary to the first functional group, and (3) a guest-terminated star polymer, wherein the guest-terminated star polymer includes a core unit, a plurality of arms extending from the core unit, and a guest molecule disposed at a terminal end of each of the plurality of arms, and wherein the host molecule and the guest-terminated star polymer are linked by one or more bonds that break under mechanical stress and reform after removal of the mechanical stress to form a mixture, wherein the contacting is performed under conditions suitable for forming a hydrophilic polymer network from the mixture.

In some embodiments, the present disclosure relates to a method of preparing a hydrogel composition for delivery of cell to a subject in need thereof, including contacting the injectable hydrogel or hydrogel of the present disclosure with a population of cells to form a hydrogel composition characterized as shear-thinning and suitable for the delivery of cells to a subject in need thereof.

In some embodiments, the present disclosure relates to a method of treating a subject in need thereof, including injecting a hydrogel of the present disclosure into a subject in need thereof. In embodiments, the hydrogel is characterized as pharmaceutically acceptable. In embodiments, the hydrogel is injected in a therapeutically acceptable amount.

The illustrative aspects of the present disclosure are designed to solve the problems herein described and/or other problems not discussed.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Embodiments of the present disclosure, briefly summarized above and discussed in greater detail below, can be understood by reference to the illustrative embodiments of the disclosure depicted in the appended drawings. However, the appended drawings illustrate only typical embodiments of the disclosure and are therefore not to be considered limiting of scope, for the disclosure may admit to other equally effective embodiments.

FIGS. 1A-1F depict schematic representations of the structures of functional supramolecular polymers (SMP) of the present disclosure such as host-guest supramolecular multi-arm PEG of the present disclosure. FIG. 1A depicts SMP-maleimide; FIG. 1B depicts SMP-thiol; FIG. 1C depicts SMP-acrylate; FIG. 1D depicts SMP-norbornene; FIG. 1E depicts SMP-stained ring; FIG. 1F depicts SMP-azide 4-arm-, 6-arm-, and 8-arm-PEG (such as PEG having a molecular weight of up to 40 kDa, or in the range of 10-40 kDa) containing guest adamantane groups at the terminals as described in this disclosure.

FIGS. 2A-2C depict a schematic representation of mono functionalization of β-cyclodextrin with different reactive groups (FIG. 2A) maleimide; (FIG. 2B) 2-iminothiolate; (FIG. 2C) N-succinimidyl acrylate.

FIG. 3 depicts a schematic representation of mono-functionalization of β-cyclodextrin with different reactive groups than FIGS. 2A-2C.

FIG. 4 depicts a schematic representation of adamantane-terminated multi-arm PEG and suitable reactive conditions for forming 8-Arm PEG-adamantane.

FIG. 5 depicts a schematic representation of functionalized hyaluronic acid (HA) employed in development of shear-thinning hydrogels. In embodiments, modified HA with different degree of substitution from 10 to 75% are also involved.

FIG. 6 depicts a schematic representation of the structures of modified heparin employed in immobilization of biochemical cues within the hydrogels. It contains either multiple repeats of a reactive moiety on the backbone or single reactive group at the reducing end or heparin.

It is noted that the drawings of the disclosure are not necessarily to scale. The drawings are intended to depict only typical aspects of the disclosure, and therefore should not be considered as limiting the scope of the disclosure. In the drawings, like numbering represents like elements between the drawings.

### DETAILED DESCRIPTION

The present disclosure provides hydrogels such as supramolecular hydrogels with controlled mechanical properties suitable for use in, inter alia, cell delivery, treatments, and 3D printing. In embodiments, the hydrogel chemistry such as supramolecular chemistry and/or host/guest polymer chemistry alters and/or tunes properties of hydrogels such as mechanical/rheological properties and/or dynamic properties such as kinetics of bond formation.

In some embodiments, the present disclosure includes an injectable hydrogel, including: a water-soluble polymer including a first functional group; a host molecule including a second functional group, wherein the second functional group is characterized as complementary to the first functional group; and a guest-terminated star polymer, wherein the guest-terminated star polymer includes a core unit, a plurality of arms extending from the core unit, and a guest molecule disposed at a terminal end of each of the plurality of arms, and wherein the host molecule and the guest-terminated star polymer are linked by one or more bonds that break under mechanical stress and reform after removal of the mechanical stress.

Advantages of the hydrogel embodiments, such as supramolecular hydrogel embodiments, of the present disclosure include: host-guest linkages with reversible association/dissociation at physiological conditions; biocompatible compositions suitable for use in cell-encapsulating and injectable delivery of cells to a subject; 3D-printing compatible compositions; host-guest complexation bonds that reduce cell death during injection (e.g., the rupture of the host-guest linkages or physical bonds reduces the disruption of cell membrane); host-guest bonds or physical bonds that are adaptable to their cellular environment and allow cell infiltration, attachment, survival, proliferation and/or migration; tunable (rheological properties) and suitable for use as scaffolds for 3D encapsulation/culture of various cells; suitable for use as scaffolds for the development of 3D tissue constructs for clinical use because of the incorporation of cell-directing cues that promote cell function, e.g., attachment, spreading, survival, proliferation, migration and/or differentiation; includes potential candidates for cell therapies (e.g. central and peripheral nerve injuries through delivery of functional myelin-forming cells). In embodiments, depending on the hydrogel chemistry and/or host guest chemistry used, properties of the hydrogels of the present disclosure can be tuned, including mechanical/rheological properties (e.g., tuned to a preselected stiffness) and dynamic properties such as kinetics of bond formation (e.g., tuned to a preselected reformation time after shear force dissociation).

### Definitions

As used in the present specification, the following words and phrases are generally intended to have the meanings as set forth below, except to the extent that the context in which they are used indicates otherwise.

As used herein, the singular forms “a”, “an”, and “the” include plural references unless the context clearly dictates otherwise. Thus, for example, references to “a compound” include the use of one or more compound(s). “A step” of a method means at least one step, and it could be one, two, three, four, five or even more method steps.

As used herein the terms “about,” “approximately,” and the like, when used in connection with a numerical variable, generally refers to the value of the variable and to all values of the variable that are within the experimental error (e.g., within the 95% confidence interval [CI 95%] for the mean) or within ±10% of the indicated value, whichever is greater.

As used herein, the terms “physical binding”, “physical bond”, and “linked by physical bonds” generally refer to the non-covalent interaction between a pair of partner molecules or portions thereof that exhibit mutual affinity or binding capacity. In embodiments, physical binding can occur such that the partners are able to interact with each other to a substantially higher degree than with other, similar substances. This specificity can result in stable complexes (e.g., host-guest polymers) that remain bound during handling step or other techniques that typically separate chemical moieties, however, in embodiments, the physical bonds may break upon being subjected to shear force.

As used herein, the term “forming a mixture” refers to the process of bringing into contact at least two distinct species such that they mix together and interact. “Forming a reaction mixture” and “contacting” refer to the process of bringing into contact at least two distinct species such that they mix together and can react, either modifying one of the initial reactants or forming a third, distinct, species, a product. It should be appreciated, however, the resulting reaction product can be produced directly from a reaction between the added reagents or from an intermediate from one or more of the added reagents which can be produced in the reaction mixture. “Conversion” and “converting” refer to a process including one or more steps wherein a species is transformed into a distinct product.

As used herein the term “gel” is intended to connote that meaning also normally associated with that term—e.g., a material including at least one solid matter that is stabilized by bonds giving it a 3D structure, but whose molecular structure/network can be infiltrated by the molecules of a liquid, where such infiltration may or may not alter the shape or dimensions of the 3D structure.

As used herein, the term “hydrogel” is intended to connote that meaning also normally associated with that term—e.g., a three-dimensional hydrophilic network including one or more hydrophilic polymers, in which water is the dispersion medium, wherein the three-dimensional hydrophilic network is capable of maintaining its structural integrity. In embodiments, hydrogels are highly swollen (e.g., and may contain 95 to 99.9% water or over 99.9% water) natural or synthetic polymers. In embodiments, hydrogels also possess a degree of flexibility very similar to natural tissue, due to their significant water content.

As used herein the term “shear-thinning” has a meaning normally associated with that term—i.e., an effect where a fluid's or gel viscosity (the measure of a fluid's or gel's resistance to flow) decreases with an increasing rate of shear stress.

As used herein the term “self-healing” in relation to hydrogels described herein refers to a characteristic of certain materials to retain an essential shape, and repair defects within its structure.

As used herein the term “polymer” is not intended to necessarily refer to a single polymer molecule; rather it is intended to connote a mixture of individual chains, the mixture having a distribution of molecular weights, as is understood by those skilled in the art. The present disclosure is not limited to any particular molecule weight distribution, provided the distribution provides a mixture suitable for the purposes described herein. In embodiments, the term “polymer” refers to a large molecule, or macromolecule, composed of many repeated subunits. The term “monomer” refers to a molecule that may bind chemically to other molecules to form a polymer. The term “copolymer” as used herein refers to a polymer derived from more than one species of monomer.

The terms “peptide,” “polypeptide,” and “protein” are used interchangeably herein, and refer to a polymeric form of amino acids of any length, which can include coded and non-coded amino acids, chemically or biochemically modified or derivatized amino acids, and polypeptides having modified peptide backbones.

The phrase “synthetic polymer” refers to polymers that are not found in nature, even if the polymers are made from naturally occurring biomaterials.

As used herein the term “natural polymer” refers to polymers that are naturally occurring. Non-limiting examples of such polymers include collagen-based materials, chitosan, hyaluronic acid and alginate.

As used herein the term “biocompatible polymer” refers to any polymer (synthetic or natural) which when in contact with cells, tissues or body or physiological fluid of an organism does not induce adverse effects such as immunological reactions and/or rejections and the like. It will be appreciated that a biocompatible polymer can also be a biodegradable polymer.

As used herein the term “biodegradable polymer” refers to a synthetic or natural polymer which can be degraded (i.e., broken down) in the physiological environment such as by enzymes, microbes, or proteins. Biodegradability depends on the availability of degradation substrates (i.e., biological materials or portion thereof which are part of the polymer), the presence of biodegrading materials (e.g., microorganisms, enzymes, proteins) and the availability of oxygen (for aerobic organisms, microorganisms or portions thereof), carbon dioxide (for anaerobic organisms, microorganisms or portions thereof) and/or other nutrients. Aliphatic polyesters, poly(amino acids), polyalkylene oxalates, polyamides, polyamido esters, poly(anhydrides), poly(beta-amino esters), polycarbonates, polyethers, polyorthoesters, polyphosphazenes, and combinations thereof are considered biodegradable. More specific examples of biodegradable polymers include, but are not limited to, collagen (e.g., Collagen I or IV), fibrin, hyaluronic acid, polylactic acid (PLA), polyglycolic acid (PGA), polycaprolactone (PCL), poly(lactide-co-glycolide) (PLGA), polydioxanone (PDO), trimethylene carbonate (TMC), polyethylene glycol (PEG), Collagen, PEG-DMA, alginate or alginic acid, chitosan polymers, or copolymers or mixtures thereof.

As used herein the term “biosorbable” refers to those polymers which are absorbed within the host body, either through a biodegradation process, or by simple dissolution in aqueous or other body fluids. Water-soluble polymers, such as poly(ethylene oxide) are included in this class of polymers.

As used herein, “normal physiological conditions”, or “wild type operating conditions”, are those conditions of temperature, pH, osmotic pressure, osmolality, oxidation and electrolyte concentration which would be considered within a normal range at the site of administration, or the site of action, in a subject.

As used herein the term “shape and dimensional stability” refers to the ability to maintain and control the substantial shape and dimensions of a delivered volume(s) of material.

As used herein, the term “star polymer” refers to a core-link-arm polymer having a central branch point or core from which three or more essentially linear, polymeric arms emanate. In embodiments, the central branch point or core may be a single atom or a chemical group having a molecular weight of about 24 to about 1,000. In embodiments, the core may include repeating core units. In embodiments, the core of a star polymer may, itself, be oligomeric or polymeric in structure. In embodiments, essentially linear, polymeric arms may have branches containing from 1 to about 20 heavy (non-hydrogen) atoms, but these branches are not formed or elongated by the same polymerization process as that forming the polymeric arms emanating from the central branch point or core. In this way, star polymers differ from hyperbranched and dendritic polymers.

As used herein, the term “supramolecular hydrogel” refers to the hydrogel whose three-dimensional networks are formed by driving forces that are non-covalent interactions.

As used herein, the term “hydrogel” refers to materials having water and three-dimensional networks with or without additional other components.

As used herein, the term “host-guest complex” refers to an entity including two or more molecules in which a host molecule contains a guest molecule, either totally or in part, using physical forces. In embodiments, a host refers to a molecular configuration that resembles the shape of a ring, with a central cavity that contains a guest molecule, either totally or in part, using only physical forces.

Before embodiments are further described, it is to be understood that this disclosure is not limited to particular embodiments described, as such may, of course, vary. It is also to be understood that the terminology used herein is for the purpose of describing particular embodiments only and is not intended to be limiting.

Where a range of values is provided, it is understood that each intervening value, to the tenth of the unit of the lower limit unless the context clearly dictates otherwise, between the upper and lower limit of that range and any other stated or intervening value in that stated range, is encompassed within the present disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

### DETAILED DESCRIPTION OF CERTAIN EMBODIMENTS

The present disclosure is directed to hydrogels such as supramolecular hydrogels or bioactive hydrogels with controlled mechanical properties suitable for use in, inter alia, cell delivery, treatments, and 3D printing. Hydrogel chemistry and/or guest/host polymer chemistry described herein alters and/or tunes properties of hydrogels such as mechanical/rheological properties and/or dynamic properties such as kinetics of bond formation and gelation time.

In embodiments, shear-thinning hydrogels and/or supramolecular hydrogels are composed of two or more chemical species such as two or more polymers (including but not limited to: natural polymers, synthetic polymers, biosorbable polymers, or biodegradable polymers) or oligomers that are held together in unique structural relationships by forces other than those of full covalent bonds. Non-covalent bonding such as linking or physical binding or physical bonds plays a role in the shear-thinning and self-healing properties of the hydrogels and/or supramolecular hydrogels of the present disclosure. In embodiments, non-covalent interactions that contribute to physical binding include host-guest complexation, hydrogen bonds, van der Waals forces, and hydrophobic interactions.

In embodiments, a shear-thinning hydrogel such as supramolecular hydrogel of the present disclosure is capable of self-assembling into a gelled network by interaction of its associated non-covalent linkages. When subjected to a mechanical shear (such as when forced to flow through a needle, catheter, or cannula), at least some of the non-covalent linkages within the hydrogel and/or supramolecular hydrogel of the present disclosure disassociate, leading to a disassembly of the gel network and a temporary thinning of the gel (lowering of the viscosity). Upon the removal of the mechanical shear force, the original gel re-assembles/recovers to a state (e.g., viscosity, stiffness, or diffusivity) the same as, or close to, its pre-shear state. Without intending to be bound by the present disclosure, this property is believed to be responsible, at least in part, for a self-healing nature of the hydrogels and/or supramolecular hydrogel. In embodiments, the present disclosure includes hydrogel and/or supramolecular hydrogel embodiments which are included those characterized as rapid healing or rapid recovery. In embodiments, the shear-thinning hydrogels and/or supramolecular hydrogels are characterized as having shape and dimensional stability.

In embodiments, the present disclosure is directed to settable, shear-thinning, self-healing gels, including hydrogels, including one or more non-covalent crosslinks (giving rise to the ability to deform and flow into liquids under shear-stress and recover back into the gels or hydrogels upon stress removal). In embodiments, hydrogels, such as supramolecular hydrogels, as described herein, exhibit a property of self-healing, as described above. In embodiments, supramolecular hydrogels include one or more chemical moieties which provide for the ability to form crosslinks, such as chemical physical crosslinks, which can then stabilize a hydrophilic network. In certain embodiments, this recovery from shear is complete within minutes or even seconds such as 10 seconds to 120 seconds.

In some embodiments the present disclosure includes a hydrogel, supramolecular hydrogel, or an injectable hydrogel, including: a water-soluble polymer including a first functional group; a host molecule including a second functional group, wherein the second functional group is characterized as complementary to the first functional group; and a guest-terminated star polymer, wherein the guest-terminated star polymer includes a core unit, a plurality of arms extending from the core unit, and a guest molecule disposed at a terminal end of each of the plurality of arms, and wherein the host molecule and the guest-terminated star polymer are linked by one or more bonds that break under mechanical stress and reform after removal of the mechanical stress. In embodiments, it should be understood the functional groups may be functional prior to reacting or forming a hydrogel of the present disclosure. In other words, the functional groups may have initially been present as a reactive functional group and reacted with or bound to a complementary functional group to form the compositions of the present disclosure.

In embodiments, the mechanical stress is a mechanical shear stress such as ejection pressure from a syringe or needle. In embodiments, the shear stress can vary depending upon whether a needle or syringe is selected for use, and the bore size of the needle or syringe. In embodiments, non-limiting examples of ejection pressure may be between 1 MPa to 25 MPa. See e.g. Wahlberg, B., Ghuman, H., Liu, J. R. et al. Ex vivo biomechanical characterization of syringe-needle ejections for intracerebral cell delivery. Sci Rep 8, 9194 (2018) (herein incorporated by reference). In embodiments, altering the percentage of constituents such as hydrophilic polymer, hyaluronic acid, or the molecular weight of PEG, the flowability of the material when subjected to shear stress can vary.

In embodiments, non-limiting examples of water-soluble polymer suitable for use herein include hyaluronic acid, gelatin, alginate, agarose, chitosan, dextran, collagen, fibronectin, or combinations thereof. In some embodiments, the water-soluble polymer is hyaluronic acid, natural hyaluronic acid, or synthetic hyaluronic acid. In embodiments, the water-soluble polymer includes a first functional group, such as —SH, NH₂, azide, ketone, aldehyde, furfuryl, maleimide, strained ring, allyl, acrylate, acrylate, alkyne, hydrazide, ketone, furfuryl, norbornene, and oxyamine, or diene, pyridyl disulfide.

In embodiments, the present disclosure includes a host molecule. A non-limiting example of a host molecule includes β-cyclodextrin or mono-functionalized β-cyclodextrin. In embodiments, the host molecule incudes a second functional group, wherein the second functional group is characterized as complementary to the first functional group of the water-soluble polymer. In embodiments, the second functional group is characterized as mono-functional and selected from the group consisting of thiol, azide, maleimide, strained ring, allyl, acrylate, acrylate, alkyne, hydrazide, ketone, aldehyde, furfuryl, norbornene, and oxyamine. In embodiments, a β-cyclodextrin is a suitable host molecule which may include a functionalized unit including one or more of maleimide, acrylate, N-succinimidyl acrylate, thiol, 2-iminothiolate, azide, strained ring, norbornene groups, or combinations thereof.

In some embodiments, the present disclosure includes a guest-terminated star polymer. In embodiments, the guest-terminated star polymer includes a core unit, a plurality of arms extending from the core unit, and a guest molecule is disposed at a terminal end of each of the plurality of arms. In embodiments, the core unit is characterized as n-arm-polyethylene glycol, wherein n is an integer between 1 to 10. In embodiments, a guest-terminated star polymer of the present disclosure includes a guest-terminated multi-arm PEG. Without wishing to be bound by the present disclosure, the inclusion of more arms extending from the core unit increases the stiffness of the product or hydrogel, compared to a product or hydrogel including less arms extending from the core unit. In embodiments, the plurality of arms is present in an amount of 4, 6, or 8. In embodiments, a plurality of arms are present in a number of 2, 4, 6, or 8 arms. In some embodiments, the plurality of arms include functional terminal ends that complement the functionalized unit. In some embodiments, the core unit is characterized as n-arm-polyethylene glycol, wherein n is an integer between 2 and 20. In embodiments, n is an integer of 2-10, and wherein the n-arm-polyethylene glycol has a molecular weight between 10-40 kDa. In embodiments, the plurality of arms include functional terminal ends suitable for attaching the guest molecule to the functional terminal ends. In some embodiments, the n-arm polyethylene glycol includes an end group including amine, hydroxyl, or carboxylic group, wherein the end group is suitable for conjugating adamantane. In some embodiments, the host and guest are present or provided and mixed in a molar ratio of 10:1 or 1:10, or 5:1 or 1:5, or about 1:1, or 1:1.

In some embodiments, a guest-terminated star polymer suitable for use herein is one or more of guest-terminated hyperbranched G2-PEG20k-OH, hyperbranched G3-PEG20k-OH, hyperbranched G4-PEG20k-OH, hyperbranched G2-PEG10k-OH, hyperbranched G3-PEG10k-OH, hyperbranched G4-PEG10k-OH, hyperbranched G2-PEG6k-OH, or hyperbranched G3-PEG6k-OH, hyperbranched G4-PEG6k-OH. In embodiments, a guest-terminated star polymer suitable for use herein is an 8-arm-PEG adamantane.

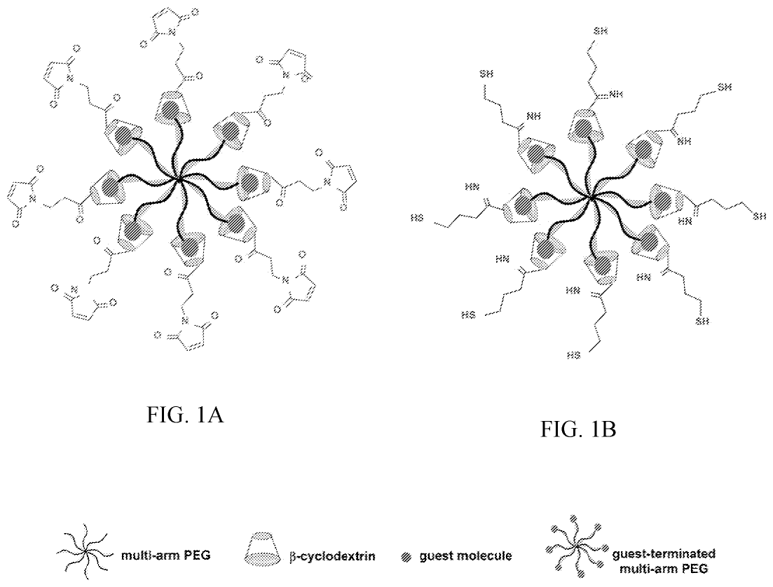

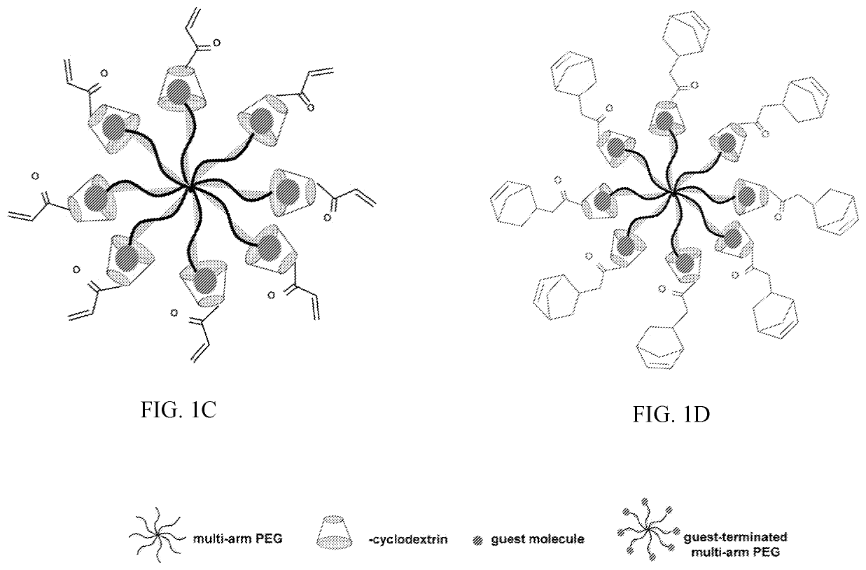

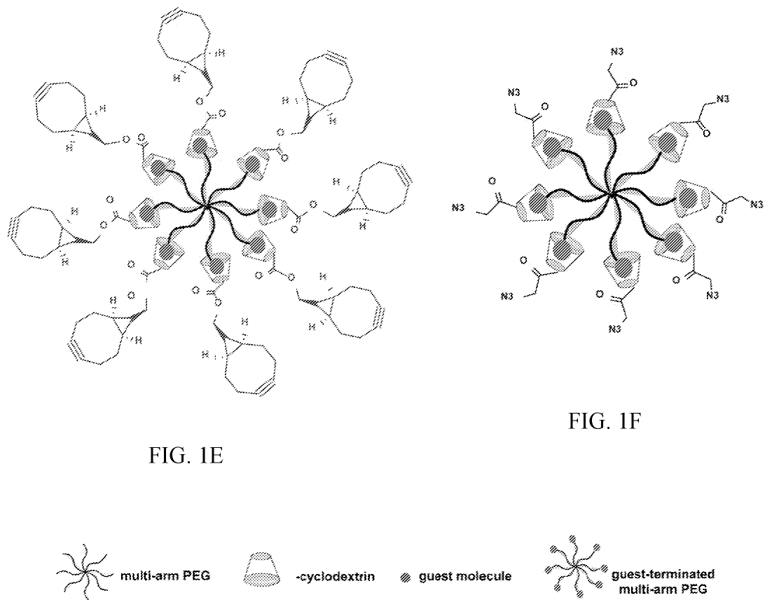

In some embodiments, a host-molecule and a guest-star polymer are physically linked to form a multi-arm supramolecular polymer (SMP). Non-limiting examples of host-molecules and guest-star polymers that physically link to form a multi-arm SMP include SMP-maleimide, SMP-thiol, SMP-acrylate, SMP-norbornene, SMP-strained ring, SMP-azide, or an n-arm-PEG including one or more adamantane groups at one or more arm terminal ends, wherein n=2-6. Referring to FIGS. 1A-1F, FIG. 1A depicts SMP-maleimide; FIG. 1B depicts SMP-thiol; FIG. 1C depicts SMP-acrylate; FIG. 1D depicts SMP-norbornene; FIG. 1E depicts SMP-stained ring; FIG. 1F depicts SMP-azide 4-arm-, 6-arm-, and 8-arm-PEG (such as PEG having a molecular weight of up to 40 kDa, or in the range of 10-40 kDa) including guest adamantane groups at the terminals as described in this disclosure.

In embodiments, the core unit is characterized as n-arm-polyethylene glycol, wherein n is an integer between 1 to 10. In embodiments, the n-arm-polyethylene glycol has a molecular weight between 10-40 kDa, and the n-arm polyethylene glycol includes an end group including carboxylic, hydroxyl, amine, acyl, acrylate, or thiol, wherein the end group is suitable for conjugating a guest molecule.

In some embodiments, the host molecule and the guest-terminated star polymer are linked by one or more bonds that break under mechanical stress and reform after removal of the mechanical stress. In some embodiments, the bonds are physical bonds are reversible at physiological conditions, such as normal physiological conditions are those of temperature, pH, osmotic pressure, osmolality, oxidation and electrolyte concentration which would be considered within a normal range at a site of administration, or at the tissue or organ at the site of action, to a subject in need thereof. In embodiments, the one or more bonds are characterized as physical bonds and further characterized as reversible under a shear-stress stimuli. In embodiments, the one or more bonds are physical bonds characterized as reversible at normal physiological conditions.

In some embodiments, the guest molecule is one or more of adamantane, ferrocene, isopropyl, azobenzene or their derivatives, any other hydrophobic compounds, and combinations thereof. In embodiments, the host molecule and guest molecule are present in a reactive group molar ratio in a range of 0.01-1.0 or 1.0-0.01.

In embodiments, an injectable hydrogel of the present disclosure flows under a shear stress and stiffens after removal of the shear stress. In embodiments, the hydrogel is characterized as having a tunable mechanical stiffness. In embodiments, the injectable hydrogel changes state from a solid to a liquid upon being subjected to shear force. In some embodiments, a cross-linking reaction provides a physically cross-linked hydrogel having a mechanical stability that is higher than the mechanical stability of the hydrogel before physical cross-linking.

In embodiments, the present disclosure includes an injectable hydrogel further including one or more immobilized biological cues. Non-limiting examples of suitable immobilized biological cues include one or more short peptides, or growth factors disposed within a hydrophilic polymer network. In some embodiments, biological cues suitable for use herein include heparin-binding proteins such as protease/esterase inhibitors, enzymes, growth factors, chemokines, pathogen proteins, and the like. Non-limiting examples of protease/esterase inhibitors include antithrombin, proteinase nexin-1, protein C inhibitor, plasminogen activator inhibitor 1, secretory leukocyte protease inhibitor, C1 inhibitor, and the like. Non-limiting examples of enzymes include factors Xa, IXa, IIa (thrombin), neutrophil elastase, cathepsin G, superoxide dismutase, and the like. Non-limiting examples of growth factors suitable for use herein include FGF, hepatocyte growth factor, and the like. Non-limiting examples of suitable chemokines for use herein include platelet growth factor 4, interleukin 8, stromal-derived factor 1a, lipid-binding proteins, and the like. Non-limiting examples of pathogen proteins include HIV-1 envelope protein glycoprotein 120, herpes simplex virus envelope proteins glycoproteins B (gB), gC and gD, dengue virus envelop protein, malaria circumsporozoite protein, and the like.

In some embodiments, the injectable hydrogel of the present disclosure includes one or more pharmaceutically active drugs or nutraceuticals, a population of cells, a peptide or peptide derivative, one or more types of nanoparticles or quantum dots, one or more fluorescent or phosphorescent materials, one or more magnetic materials, or a combination thereof. In embodiments, the injectable hydrogel is characterized as pharmaceutically acceptable.

In some embodiments, the present disclosure relates to a shear-thinning supramolecular hydrogel, including: a water-soluble polymer including a first functional group; a host molecule including a second functional group, wherein the second functional group is characterized as complementary to the first functional group; and a guest-terminated star polymer, wherein the guest-terminated star polymer includes a core unit, a plurality of arms extending from the core unit, and a guest molecule disposed at a terminal end of each of the plurality of arms, and wherein the host molecule and the guest-terminated star polymer are linked by one or more bonds that break under mechanical stress and reform after removal of the mechanical stress. In embodiments, the first functional group is —SH, NH₂, azide, ketone, aldehyde, furfuryl, maleimide, strained ring, allyl, acrylate, acrylate, alkyne, hydrazide, ketone, furfuryl, norbornene, and oxyamine, or diene, pyridyl disulfide. In embodiments, the second functional group is characterized as mono-functional and selected from the group consisting of thiol, azide, maleimide, strained ring, allyl, acrylate, acrylate, alkyne, hydrazide, ketone, aldehyde, furfuryl, norbornene, and oxyamine. In some embodiments, the guest molecule is selected from the group consisting of: adamantane, ferrocene, isopropyl, azobenzene or their derivatives, any other hydrophobic compounds, and combinations thereof. In embodiments, the host molecule is β-cyclodextrin or cucurbit[8]uril.

In embodiments, non-limiting examples of water-soluble polymer suitable for use in a shear-thinning supramolecular hydrogel include hyaluronic acid, gelatin, alginate, agarose, chitosan, dextran, collagen, fibronectin, or combinations thereof. In some embodiments, the water-soluble polymer is hyaluronic acid, natural hyaluronic acid, or synthetic hyaluronic acid. In embodiments, the water-soluble polymer includes a first functional group, such as —SH, NH₂, azide, ketone, aldehyde, furfuryl, maleimide, strained ring, allyl, acrylate, acrylate, alkyne, hydrazide, ketone, furfuryl, norbornene, and oxyamine, or diene, pyridyl disulfide.

In embodiments, the shear-thinning supramolecular hydrogel includes a host molecule. A non-limiting example of a host molecule includes β-cyclodextrin or mono-functionalized β-cyclodextrin. In embodiments, the host molecule incudes a second functional group, wherein the second functional group is characterized as complementary to the first functional group of the water-soluble polymer. In embodiments, the second functional group is characterized as mono-functional and selected from the group consisting of thiol, azide, maleimide, strained ring, allyl, acrylate, acrylate, alkyne, hydrazide, ketone, aldehyde, furfuryl, norbornene, and oxyamine. In embodiments, a β-cyclodextrin is a suitable host molecule which may include a functionalized unit including one or more of maleimide, acrylate, N-succinimidyl acrylate, thiol, 2-iminothiolate, azide, strained ring, norbornene groups, or combinations thereof.

In embodiments, the shear-thinning supramolecular hydrogel includes a guest-terminated star polymer such as those described above including a core unit, a plurality of arms extending from the core unit, and a guest molecule is disposed at a terminal end of each of the plurality of arms. In embodiments, the core unit is characterized as n-arm-polyethylene glycol, wherein n is an integer between 1 to 10. In embodiments, a guest-terminated star polymer of the present disclosure includes a guest-terminated multi-arm PEG. In embodiments, the plurality of arms is present in an amount of 4, 6, or 8. In embodiments, a plurality of arms are present in a number of 2, 4, 6, or 8 arms. In some embodiments, the plurality of arms include functional terminal ends that complement the functionalized unit. In some embodiments, the core unit is characterized as n-arm-polyethylene glycol, wherein n is an integer between 2 and 20. In embodiments, n is an integer of 2-10, and wherein the n-arm-polyethylene glycol has a molecular weight between 10-40 kDa. In embodiments, the plurality of arms include functional terminal ends suitable for attaching the guest molecule to the functional terminal ends. In some embodiments, the n-arm polyethylene glycol includes an end group including amine, hydroxyl, or carboxylic group, wherein the end group is suitable for conjugating adamantane. In some embodiments, the host and guest are present or provided and mixed in a molar ratio of 10:1 or 1:10, or 5:1 or 1:5, or about 1:1, or 1:1.

In some embodiments, the shear-thinning supramolecular hydrogel of the present disclosure includes guest-terminated multi-arm PEG molecules, including a plurality of arms, wherein each of the plurality of arms includes a functional terminal end suitable for attaching the guest molecule to the functional terminal ends.

In some embodiments, the present disclosure relates to a shear-thinning supramolecular hydrogel, including: a water-soluble polymer including a first functional group; a host molecule including a second functional group, wherein the second functional group is characterized as complementary to the first functional group; and a guest-terminated star polymer, wherein the guest-terminated star polymer includes a core unit, a plurality of arms extending from the core unit, and a guest molecule disposed at a terminal end of each of the plurality of arms, and wherein the host molecule and the guest-terminated star polymer are linked by one or more bonds that break under mechanical stress and reform after removal of the mechanical stress. In some embodiments, the host molecule and guest molecule are present in a reactive group molar ratio in a range of 0.01-1.0 or 1.0-0.01. In some embodiments, the host-molecule and guest-star polymer physically link to form a multi-arm supramolecular polymer (SMP)-maleimide, SMP-thiol, SMP-acrylate, SMP-norbornene, SMP-strained ring, SMP-azide, or an n-arm-PEG including one or more adamantane groups at one or more arm terminal ends, wherein n=2-6. In some embodiments, the one or more bonds are characterized as physical bonds and further characterized as reversible under a shear-stress stimuli. In embodiments, the hydrogel is characterized as having a tunable mechanical stiffness. In embodiments, the shear-thinning supramolecular hydrogel further includes one or more immobilized biological cues such as those described above. In embodiments, the the immobilized biological cues include one or more short peptides, growth factors disposed within a hydrophilic polymer network. In embodiments, the shear-thinning supramolecular hydrogel further includes one or more pharmaceutically active drugs or nutraceuticals, a population of cells, a peptide or peptide derivative, one or more types of nanoparticles or quantum dots, one or more fluorescent or phosphorescent materials, one or more magnetic materials, or a combination thereof. In embodiments, the sheer-thinning hydrogel changes state from a solid to a liquid upon being subjected to shear force. In some embodiments, a cross-linking reaction provides a physically cross-linked hydrogel having a mechanical stability that is higher than the mechanical stability of the hydrogel before physical cross-linking.

In some embodiments, the present disclosure relates to a shear-thinning supramolecular hydrogel, including: a water-soluble polymer including a first functional group; a host molecule including a second functional group, wherein the second functional group is characterized as complementary to the first functional group; and a guest-terminated star polymer, wherein the guest-terminated star polymer includes a core unit, a plurality of arms extending from the core unit, and a guest molecule disposed at a terminal end of each of the plurality of arms, and wherein the host molecule and the guest-terminated star polymer are linked by one or more bonds that break under mechanical stress and reform after removal of the mechanical stress. In some embodiments, the guest-terminated star polymer is an 8-arm-PEG adamantane. In some embodiments, non-limiting examples of water-soluble polymer suitable for use herein includes hyaluronic acid, gelatin, alginate, agarose, chitosan, dextran, collagen, fibronectin, or combinations thereof. In some embodiments, non-limiting examples of guest-terminated polymer includes guest-terminated hyperbranched G2-PEG20k-OH, hyperbranched G3-PEG20k-OH, hyperbranched G4-PEG20k-OH, hyperbranched G2-PEG10k-OH, hyperbranched G3-PEG10k-OH, hyperbranched G4-PEG10k-OH, hyperbranched G2-PEG6k-OH, or hyperbranched G3-PEG6k-OH, hyperbranched G4-PEG6k-OH.

In some embodiments, the present disclosure relates to hydrogel, including: a water-soluble polymer including a first functional group; a host molecule including a second functional group, wherein the second functional group is characterized as complementary to the first functional group; and a guest-terminated star polymer, wherein the guest-terminated star polymer comprises a core unit, a plurality of arms extending from the core unit, and a guest molecule disposed at a terminal end of each of the plurality of arms, and wherein the host molecule and the guest-terminated star polymer are linked by one or more bonds that break under mechanical stress and reform after removal of the mechanical stress, wherein the guest-terminated star polymer is an n-arm-PEG adamantane, wherein n is an integer characterized as 2, 4, 6, or 8.

In some embodiments, the present disclosure includes a bioactive hydrogel, wherein the bioactive hydrogel is prepared by: contacting a water-soluble polymer including a first functional group, a host molecule including a second functional group, wherein the second functional group is characterized as complementary to the first functional group, and a guest-terminated star polymer, wherein the guest-terminated star polymer comprises a core unit, a plurality of arms extending from the core unit, and a guest molecule disposed at a terminal end of each of the plurality of arms, and wherein the host molecule and the guest-terminated star polymer are linked by one or more bonds that break under mechanical stress and reform after removal of the mechanical stress to form a mixture, wherein the contacting is performed under conditions suitable for forming a hydrophilic polymer network from the mixture. In embodiments, non-limiting examples of suitable water-soluble polymer includes one or more of hyaluronic acid, gelatin, alginate, agarose, chitosan, dextran, collagen, fibronectin, or combinations thereof. In embodiments, non-limiting examples of suitable guest-terminated polymer includes one or more of guest-terminated hyperbranched G2-PEG20k-OH, hyperbranched G3-PEG20k-OH, hyperbranched G4-PEG20k-OH, hyperbranched G2-PEG10k-OH, hyperbranched G3-PEG10k-OH, hyperbranched G4-PEG10k-OH, hyperbranched G2-PEG6k-OH, or hyperbranched G3-PEG6k-OH, hyperbranched G4-PEG6k-OH, or combinations thereof. In embodiments, the bioactive hydrogel is further prepared by contacting the mixture with one or more immobilized biological cues. In embodiments, the immobilized biological cues include one or more short peptides, growth factors disposed within a hydrophilic polymer network. In some embodiments, the mixture further includes one or more pharmaceutically active drugs or nutraceuticals, a population of cells; a peptide or peptide derivative, one or more types of nanoparticles or quantum dots, one or more fluorescent or phosphorescent materials, one or more magnetic materials, or a combination thereof. In some embodiments, a bioactive hydrogel further includes a protein including a heparin binding domain and a cell binding domain, wherein the heparin-binding domain enables binding to functionalized heparin, and wherein the functionalized heparin includes a functional thiol group. In embodiments, the protein is a heparin binding growth factor or a fusion-heparin-binding protein. In some embodiments, the heparin binding growth factor comprises PDGF-AA, PDGF-AB, PDGF-BB, PDGF-C, PDGF-D, FGF2, NRG1, VEGF, HGF, FGF-1, FGF-2, FGF-7, FGF-9, FGF-10, FGF-13, FGF1, FGF2, FGF7, or the like.

In some embodiments, the bioactive hydrogel of the present disclosure includes immobilized biological cues include one or more short peptides, or growth factors disposed within a hydrophilic polymer network. In some embodiments, biological cues suitable for use herein include heparin-binding proteins such as protease/esterase inhibitors, enzymes, growth factors, chemokines, pathogen proteins, and the like. Non-limiting examples of protease/esterase inhibitors include antithrombin, proteinase nexin-1, protein C inhibitor, plasminogen activator inhibitor 1, secretory leukocyte protease inhibitor, C1 inhibitor, and the like. Non-limiting examples of enzymes include factors Xa, IXa, IIa (thrombin), neutrophil elastase, cathepsin G, superoxide dismutase, and the like. Non-limiting examples of growth factors suitable for use herein include FGF, hepatocyte growth factor, and the like. Non-limiting examples of suitable chemokines for use herein include platelet growth factor 4, interleukin 8, stromal-derived factor 1a, lipid-binding proteins, and the like. Non-limiting examples of pathogen proteins include HIV-1 envelope protein glycoprotein 120, herpes simplex virus envelope proteins glycoproteins B (gB), gC and gD, dengue virus envelop protein, malaria circumsporozoite protein, and the like.

In embodiments, the bioactive hydrogel of the present disclosure includes a fusion-heparin-binding protein including HBD-(REDV)n, HBD-(RGDS)n, where n=number of peptide repeats (typically from 3-5). In some embodiments, the bioactive hydrogel is further contacted with cells and/or bioactive molecules, or short peptides with reactive groups (REDV-SH, RGD-SH, IKVAV-SH) for chemical attachment within the hydrogel.

In some embodiments, the bioactive hydrogel of the present disclosure, further includes one or more thermo-responsive polymers with transition temperatures around 37° C.

In some embodiments, the present disclosure includes a method of making a hydrogel, including: contacting (1) a water-soluble polymer including a first functional group, (2) a host molecule including a second functional group, wherein the second functional group is characterized as complementary to the first functional group, and (3) a guest-terminated star polymer, wherein the guest-terminated star polymer includes a core unit, a plurality of arms extending from the core unit, and a guest molecule disposed at a terminal end of each of the plurality of arms, and wherein the host molecule and the guest-terminated star polymer are linked by one or more bonds that break under mechanical stress and reform after removal of the mechanical stress to form a mixture, wherein the contacting is performed under conditions suitable for forming a hydrophilic polymer network from the mixture. In embodiments, the constituents (1), (2), and (3) are mixed sequentially, or simultaneously. In some embodiments a first functional group, is one or more of —SH, NH₂, azide, ketone, aldehyde, furfuryl, maleimide, strained ring, allyl, acrylate, acrylate, alkyne, hydrazide, ketone, furfuryl, norbornene, and oxyamine, or diene, pyridyl disulfide. In some embodiments, the second functional group is one or more of thiol, azide, maleimide, strained ring, allyl, acrylate, acrylate, alkyne, hydrazide, ketone, aldehyde, furfuryl, norbornene, and oxyamine.

In embodiments, the water-soluble polymer includes those described above such as one or more of hyaluronic acid, gelatin, alginate, agarose, chitosan, dextran, collagen, fibronectin, or combinations thereof. In embodiments, the water-soluble polymer includes hyaluronic acid and the star polymers include PEG, wherein the water-soluble polymer and the PEG are provided at a reactive groups molar ratio in the range of 0.01-1.0 or 1-0.01 and mixed at pH 7.4, about 25 degrees C. or 37 degrees C. In embodiments, a hydrogel is formed with a mechanical stiffness. In embodiments, a resulting hydrogel possesses mechanical stiffness in a range between 3 Pa and 50 kPa when measured by a dynamic oscillatory rheology. In embodiments, the present disclosure includes adding water, or adding water to the balance in an amount sufficient to form the hydrogel in a predetermined size or shape.

In embodiments, non-limiting suitable guest-terminated polymers include those described above, including guest-terminated hyperbranched G2-PEG20k-OH, hyperbranched G3-PEG20k-OH, hyperbranched G4-PEG20k-OH, hyperbranched G2-PEG10k-OH, hyperbranched G3-PEG10k-OH, hyperbranched G4-PEG10k-OH, hyperbranched G2-PEG6k-OH, or hyperbranched G3-PEG6k-OH, hyperbranched G4-PEG6k-OH, or combinations thereof. In embodiments, the method of making a hydrogel further includes contacting the mixture with one or more immobilized biological cues such as those described above, including one or more short peptides, growth factors disposed within a hydrophilic polymer network. In some embodiments, the method of making a hydrogel further includes adding to the mixture one or more pharmaceutically active drugs or nutraceuticals, a population of cells, a peptide or peptide derivative, one or more types of nanoparticles or quantum dots, one or more fluorescent or phosphorescent materials, one or more magnetic materials, or a combination thereof. In some embodiments, the method of making a hydrogel further includes adding to the mixture a protein including a heparin binding domain and a cell binding domain to the mixture, wherein the heparin-binding domain enables binding to functionalized heparin, and wherein the functionalized heparin includes a functional thiol group. In some embodiments, the protein is a heparin binding growth factor or a fusion-heparin-binding protein. In some embodiments, the heparin binding growth factor includes PDGF-AA, PDGF-AB, PDGF-BB, PDGF-C, PDGF-D, FGF2, NRG1, VEGF, HGF, FGF-1, FGF-2, FGF-7, FGF-9, FGF-10, FGF-13, FGF1, FGF2, FGF7, or the like. In some embodiments, the fusion-heparin-binding protein includes HBD-(REDV)n, HBD-(RGDS)n, where n=number of peptide repeats (typically from 3-5). In some embodiments, the hydrogel is further contacted with cells and/or bioactive molecules, or short peptides with reactive groups (REDV-SH, RGD-SH, IKVAV-SH) for chemical attachment within the hydrogel. In some embodiments, the method of making a hydrogel includes adding thermo-responsive polymers with transition temperatures around 37 degrees Celsius.

In some embodiments, the present disclosure includes a method of preparing a hydrogel composition for delivery of cell to a subject in need thereof, including contacting the injectable hydrogel of the present disclosure with a population of cells to form a hydrogel composition characterized as shear-thinning and suitable for the delivery of cells to a subject in need thereof. In some embodiments, the hydrogel composition includes one or more water-soluble polymers such as one or more of hyaluronic acid, HA-thiol, HA-maleimide, HA-acrylate, and combinations thereof. In embodiments, the hydrogel includes a guest molecule is selected from the group consisting of: adamantane, ferrocene, isopropyl, azobenzene or their derivatives, any other hydrophobic compounds, and combinations thereof. In some embodiments, the hydrogel includes a host molecule such as β-cyclodextrin or mono-functionalized β-cyclodextrin. In embodiments, a plurality of arms are present in a number of 2, 4, 6, or 8 arms. In embodiments, the hydrogel includes one or more bonds such as physical bonds characterized as reversible at normal physiological conditions. In embodiments, the hydrogel flows under a shear stress and stiffens after removal of the shear stress. In embodiments, the cells include any cell type including pluripotent stem cells and any cell derived from them, mesenchymal stem cells, neural crest stem cells (NCSC), myelin-forming cells, Schwann cells, oligodendrocytes, neurons, and combinations thereof.

In embodiments, the present disclosure includes a method of preparing a hydrogel composition for delivery of cell to a subject in need thereof, including: contacting a population of cells with a water-soluble polymer selected from the group consisting of: hyaluronic acid, gelatin, alginate, agarose, chitosan, dextran, collagen, fibrinogen, and combinations thereof, wherein the water-soluble polymer has a functional group selected from the group consisting of thiol, amino, hydrazide, and combinations thereof to form a water-soluble polymer cell mixture; and contacting the water-soluble polymer cell mixture with a multi-armed polymer including a host component including a mono-functionalized β-cyclodextrin and an end-terminated multi-arm poly(ethylene glycol) (PEG) to form a hydrogel composition characterized as shear-thinning and suitable for the delivery of cells to a subject in need thereof. In embodiments, the water-soluble polymer is one or more of HA-thiol, HA-maleimide, and HA-acrylate. In embodiments, the cells include one or more neural crest stem cells (NCSC), myelin-forming cells, Schwann cells, oligodendrocytes, neurons, or combinations thereof.

In some embodiments, the present disclosure includes a method of treating a subject in need thereof, including injecting a hydrogel of the present disclosure into a subject in need thereof. In embodiments, the hydrogel is characterized as pharmaceutically acceptable. In embodiments, the hydrogel is injected in a therapeutically acceptable amount. In embodiments, the hydrogel includes one or more active agents in an amount sufficient to be characterized as a therapeutically acceptable amount.

In embodiments, the present disclosure includes a method of treating a subject in need thereof, including: injecting a hydrogel into a subject in need thereof, wherein the hydrogel includes a hydrophilic polymer network, including: a first water-soluble polymer including a functionalized unit; and a second star polymer including a core unit and a plurality of arms extending from the core unit, wherein the first water-soluble polymer and the second star polymer are linked by physical bonds that break under shear stress and reform after removal of the shear stress. In embodiments, the hydrogels and supramolecular hydrogels may be applied in-vivo and/or ex-vivo. Various embodiments provide that the settable or cured hydrogels are adapted to be medically acceptable for use in a mammal, including those where the mammal is a human. Such embodiments include those where the materials are at least biocompatible, pharmaceutically acceptable, or approved by the United States Food and Drug Administration in the United States (or a corresponding regulatory agency in other countries).

In embodiments, the hydrogels and supramolecular hydrogels of the present disclosure may be used to control encapsulated cell behavior, improve delivered cell retention, and control cellular release rates. These the hydrogels and supramolecular hydrogels materials can also be used to tune encapsulated drug release profiles and pharmacokinetics.

Additional exemplary applications of the hydrogels and supramolecular hydrogels include use in: scaffolds in tissue engineering; vehicles for cell encapsulation and delivery; sustained- or controlled release drug delivery systems; biosensors, including those responsive to specific molecules, such as glucose or antigens; contact lenses; adhesives, including medical and electronic adhesives biosealants; dressings for healing of burn or other hard-to-heal wounds; and reservoirs in topical drug delivery; particularly ionic drugs, delivered by iontophoresis In embodiments, the hydrogels and supramolecular hydrogels are suitable for in situ delivery e.g., at the wound site, and conform to the shape of the body where the wound such as a burn is. Since skin is not necessarily flat and may include wound areas of complicated geometry e.g., face, arms etc., in situ application of the hydrogels of the present disclosure is encompassed by this disclosure.

Certain embodiments also provide methods of preparing a controlled or sustained release formulation of a pharmaceutically active drug, nutraceutical, cell population, or particle array in a patient, each method including introducing into the patient a composition including a settable, shear-thinning hydrogel or supramolecular hydrogel as described herein, and a pharmaceutically active drug, nutraceutical, cell population, or particle. Other embodiments further comprise triggering at least one chemical covalent crosslinking reaction.

Other independent embodiments provide methods of preparing a controlled release formulation of a pharmaceutically active drug, nutraceutical, or cell population in a patient, each method including introducing into a patient the composition including (a) a settable shear-thinning hydrogel or supramolecular hydrogel of the present disclosure; and (b) a pharmaceutically active drug, nutraceutical, or cell population. Other embodiments further comprise triggering at least one chemical covalent crosslinking reaction.

Other independent embodiments provide methods of printing 3-D structures using supramolecular hydrogels, and the structures which result therefrom. In embodiments, the hydrogels or supramolecular hydrogels are useful for printing of ink such as supramolecular ink into a supramolecular support, such as shown and described in U.S. Pat. No. 10,828,399 (herein incorporated by reference).

In embodiments, the present disclosure includes a chemical synthesis methods and composition of novel injectable supramolecular hydrogels incorporating reversible host-guest linkages to render the hydrogels dynamic and shear-thinning. In embodiments, the method includes two components system, each component including one or more functional groups or reactive motifs that are complementary to each other. In embodiments, a first component includes a functional multi-arm supramolecular polymer (SMP); and a second component includes a functional water-soluble polymer.

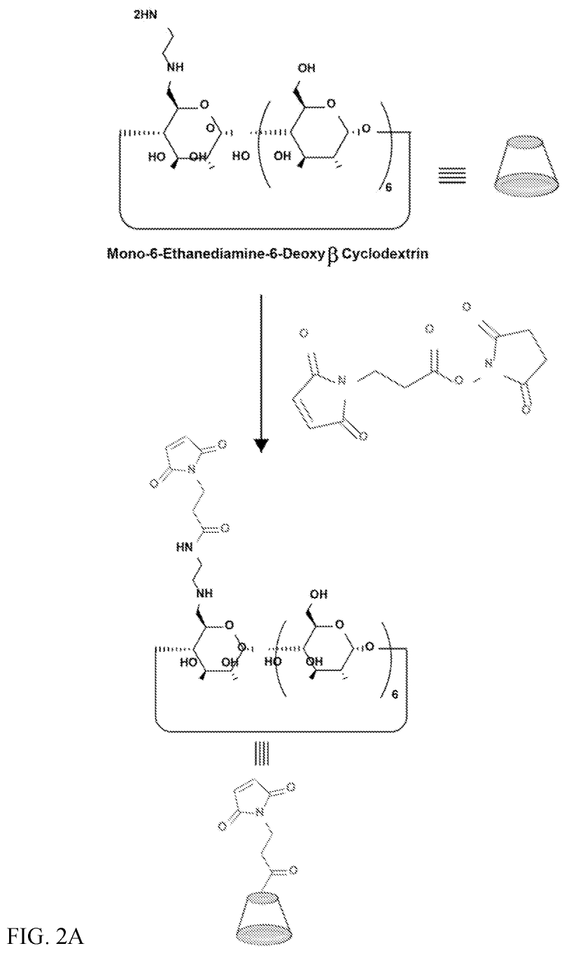

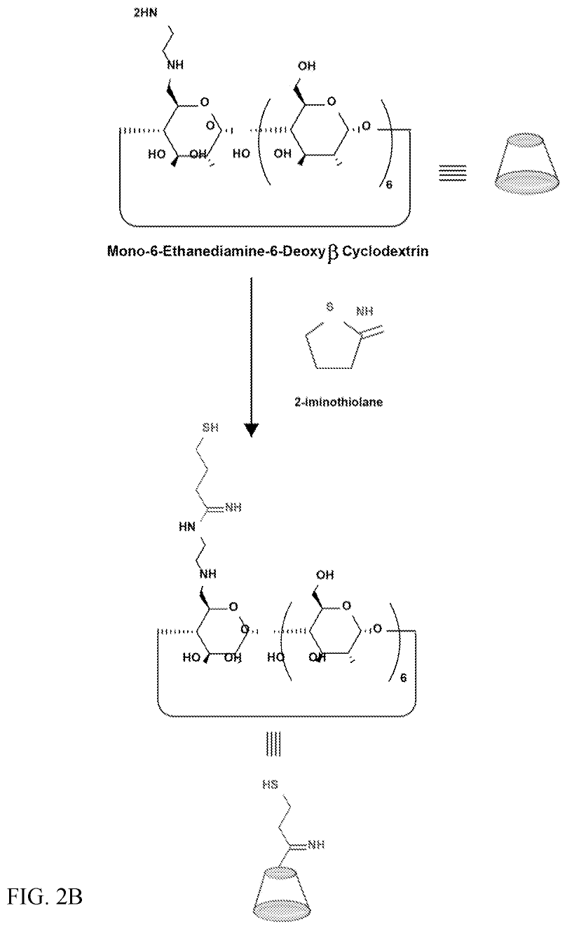

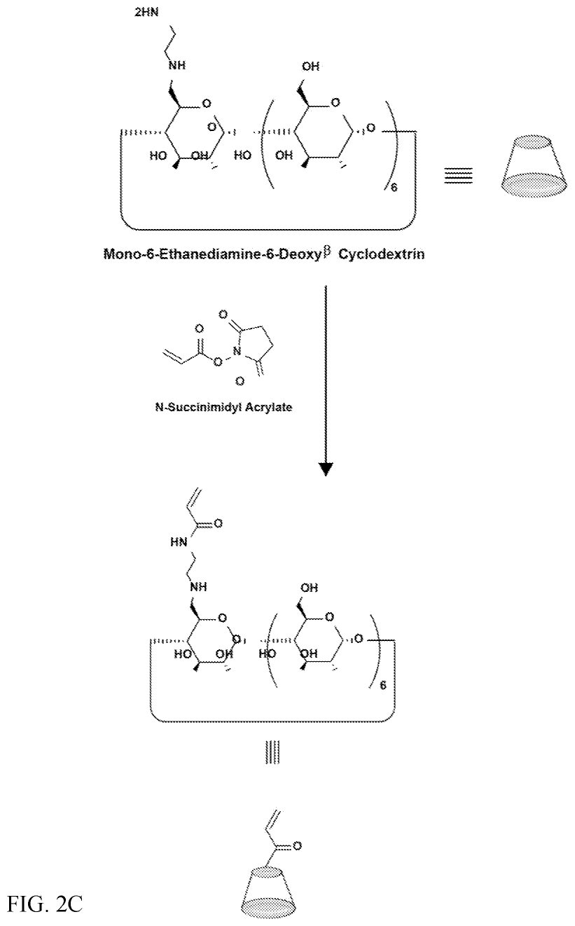

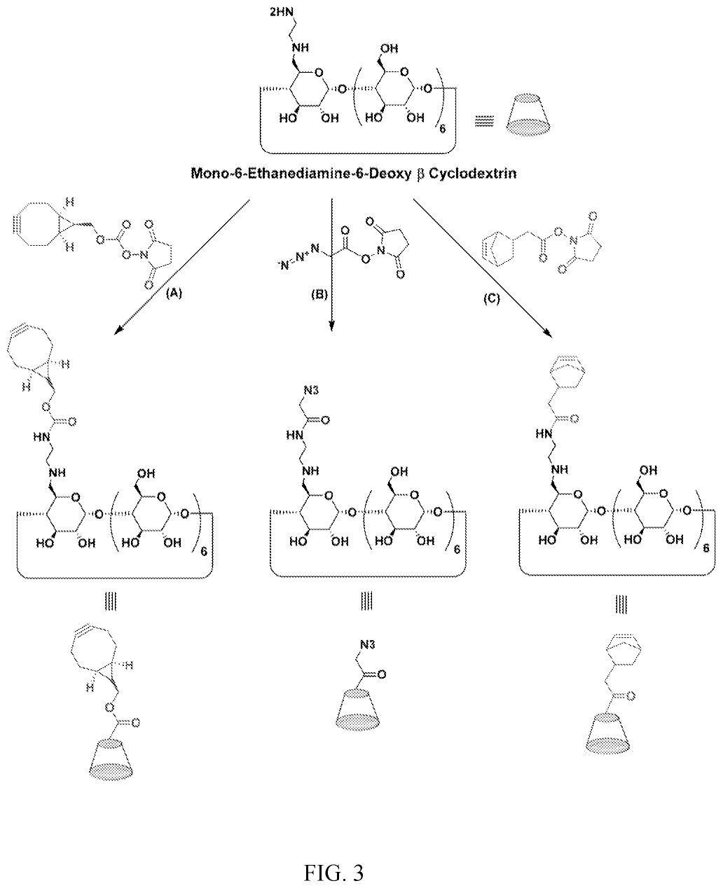

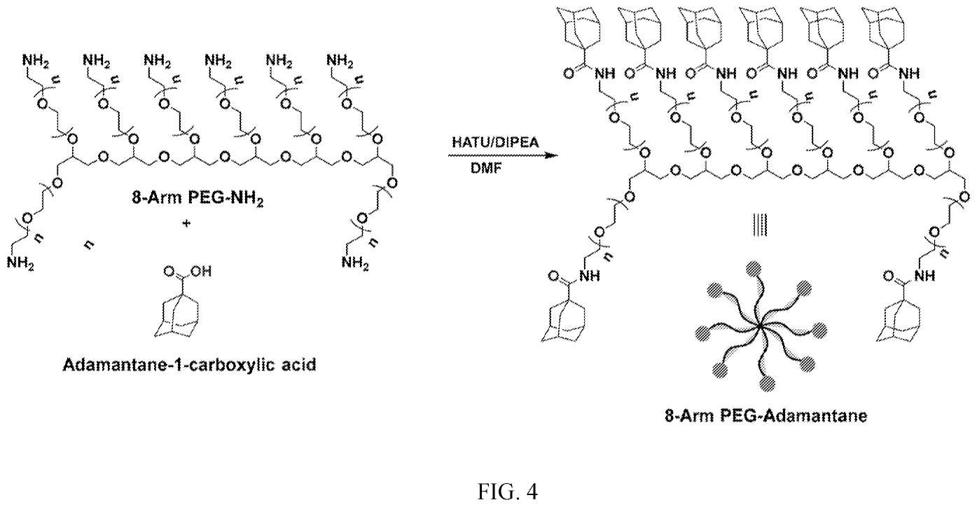

Referring now to FIGS. 1A-1F, FIGS. 1A-1F depict schematic representations of the structures of functional supramolecular polymers (SMP) of the present disclosure. FIG. 1A depicts SMP-maleimide; FIG. 1B depicts SMP-thiol; FIG. 1C depicts SMP-acrylate; FIG. 1D depicts SMP-norbornene; FIG. 1E depicts SMP-stained ring; FIG. 1F depicts SMP-azide 4-arm-, 6-arm-, and 8-arm-PEG (MW: 10-40 kDa) containing guest adamantane groups at the terminals as described in this disclosure. Here, the SMP includes a first component characterized as a mono-functionalized β-cyclodextrin (see e.g., host component in FIG. 2 (and FIG. 3); and an adamantane-terminated multi-arm poly(ethylene glycol) (PEG) (guest component, See e.g., FIG. 4).

More specifically, FIGS. 2A-2C depict a schematic representation of mono functionalization of β-cyclodextrin with different reactive groups (FIG. 2A) maleimide; (FIG. 2B) 2-iminothiolate; (FIG. 2C) N-Succinimidyl Acrylate. FIG. 3 depicts a schematic representation of mono-functionalization of β-cyclodextrin with different reactive groups than FIG. 2A-2C. FIG. 4 depicts a schematic representation of adamantane-terminated multi-arm PEG.

In embodiments, the development of the functional SMP involves three steps: a) functionalization of β-cyclodextrin with reactive group of interest (e.g. maleimide); b) modification of multi-arm PEG with a guest molecule (e.g. adamantane); and c) mixing the host and guest components at equal molar ratios of the host/guest functionalities.

In some embodiments, four-arm PEG, six-arm PEG or eight-arm PEG (core: tripentaerythritol, hexaglycerol) of different molecular weights (10-40 kDa), and different end groups (amine, hydroxyl, thiols, carboxylic, and acyl) are employed in the design of the guest component of the SMP. In other embodiments, β-cyclodextrin mono-functionalized with acrylate, maleimide, thiol, azide, strained ring, or norbornene groups are used as the host component of the functional SMP, (See e.g., FIGS. 2-3).

In some embodiments, a hydrogel formation method of the present disclosure involves three steps: 1) functionalization of water-soluble polymer with a functional group of interest (for example, thiolated-hyaluronic acid (HA-thiol)); 2) development of functional SMP incorporating complementary functional groups at terminals (for example 8-arm SMP-maleimide); and 3) mixing polymers (1) and (2) above (for example HA-thiol and SMP-maleimide) at different weight ratios in pH 7.4 to obtain hydrogels with broad range of mechanical stiffness. See e.g., TABLE 1 depicting representative examples of the compositions of supramolecular shear-thinning hydrogel developed by mixing HA-thiol (degree of substitution 16%) with functional supramolecular polymer (SMP, 8-arm, MW: 40 kDa).

TABLE 1

| | | | |
|---|---|---|---|
| HA_{1 wt %} SMP_{5 wt %} | HA_{1.5 wt %} SMP_{5 wt %} | HA_{2 wt %} SMP_{5 wt %} | HA_{2.5 wt %} SMP_{5 wt %} |
| HA_{1 wt %} SMP_{7.5 wt %} | HA_{1.5 wt %} SMP_{7.5 wt %} | HA_{2 wt %} SMP_{7.5 wt %} | HA_{2.5 wt %} SMP_{7.5 wt %} |
| HA_{1 wt %} SMP_{10 wt %} | HA_{1.5 wt %} SMP_{10 wt %} | HA_{2 wt %} SMP_{10 wt %} | HA_{2.5 wt %} SMP_{10 wt %} |
| HA_{1 wt %} SMP_{12.5 wt %} | HA_{1.5 wt %} SMP_{12.5 wt %} | HA_{2 wt %} SMP_{12.5 wt %} | HA_{2.5 wt %} SMP_{12.5 wt %} |

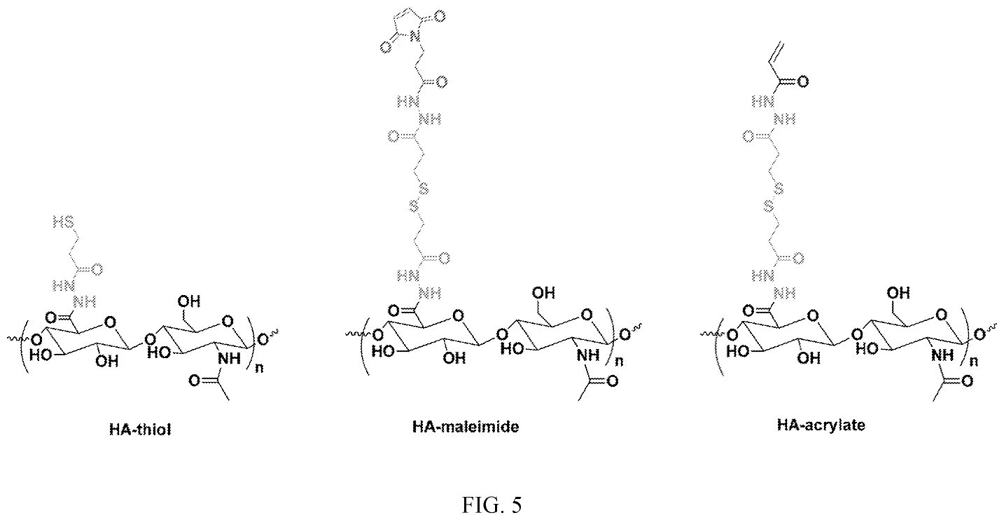

In embodiments, hyaluronic acid decorated with thiol, amino, and hydrazide groups at different degree of functionalization (10-75%) are employed in this disclosure (FIG. 5). FIG. 5 depicts a schematic representation of functionalized hyaluronic acid employed in development of shear-thinning hydrogels. Modified HA with different degree of substitution from 10 to 75% are also involved. In addition, other non-limiting examples of water-soluble polymers such as gelatin, fibrinogen, collagen, alginate, agarose, chitosan, and dextran are also included in this disclosure as a replacement of HA. Of note, when HA-thiol is used, a blocking agent is employed to block the excess thiol groups and prevent formation of disulfide bonds. In embodiments, any water-soluble polymer either synthetic, protein- or, sugar-based could be included in the compositions of the present disclosure.

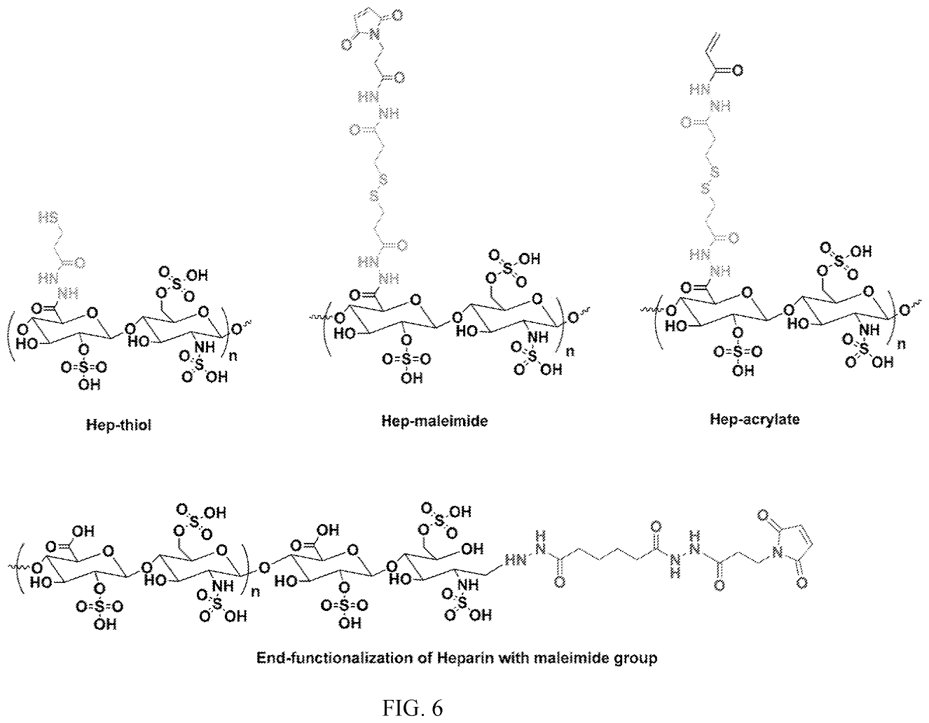

In some embodiments, the present disclosure is directed to a method and composition for development of bioactive, injectable shear-thinning hydrogel incorporating biomimetic cues to trigger specific cell responses. In embodiments, a method encompasses four components system including: 1) functional SMP with a reactive functionality; 2) HA containing a complementary reactive moiety; 3) Heparin (Hep) incorporating the same complementary motifs as HA (See e.g., FIG. 6 which depicts a schematic representation of the structures of modified heparin employed in immobilization of biochemical cues within the hydrogels. It contains either multiple repeats of a reactive moiety on the backbone or single reactive group at the reducing end or heparin); and 4) biomimetic cues of interest such as heparin binding growth factors and fusion heparin-binding proteins prepared in our lab. In embodiments, the latter are composed of at least two domains: (i) heparin binding domain (HBD) that binds thiolated or otherwise modified heparin (See. e.g., FIG. 6); and (ii) cell binding domain to trigger a certain cell behavior. In some embodiments a process sequence for development of bioactive hydrogels involves: 1) preparation of functionalized HA; 2) development of functional SMP; 3) preparation of modified Hep; 4) preparation of biochemical cues of interest that contain reactive sites for their immobilization within a hydrogel; and 5) mixing the four components at pre-defined weight ratios to obtain bioactive hydrogels with required stiffness.

In some embodiments, the present disclosure includes control of biochemical signal presentation. For example, the way biochemical cues are presented to the cells can be controlled by controlling their binding to heparin. To this end, the functionalized heparin in this disclosure includes Hep modified either only at the end-group or at multiple-sites (See e.g., FIG. 6), which may determine the degree and orientation of biochemical factor binding and presentation to the cells.

In embodiments, the biochemical cues employed in the disclosure comprise: 1) short peptides with reactive groups (e.g., RGD-SH, IKVAV-SH, REDV-SH) for chemical attachment within the hydrogels; 2) recombinant fusion proteins containing at least two domains: (i) heparin binding domain (HBD) that binds thiolated or otherwise modified heparin (See e.g., FIG. 6); and (ii) cell binding domain or multiple domains to trigger a certain cell behavior, such as a peptide that may promote cell attachment. Such fusion proteins include but are not limited to HBD-(REDV)ₙ, HBD-(RGDS)ₙ, where n=number of peptide repeats; 3) heparin binding growth factors such as PDGF-AA, PDGF-AB, PDGF-BB, PDGF-C, PDGF-D, FGF2, NRG1 and others.

Other non-limiting additives include heparin-binding proteins such as protease/esterase inhibitors, enzymes, growth factors, chemokines, pathogen proteins, and the like. Non-limiting examples of protease/esterase inhibitors include antithrombin, proteinase nexin-1, protein C inhibitor, plasminogen activator inhibitor 1, secretory leukocyte protease inhibitor, C1 inhibitor, and the like. Non-limiting examples of enzymes include factors Xa, IXa, IIa (thrombin), neutrophil elastase, cathepsin G, superoxide dismutase, and the like. Non-limiting examples of growth factors suitable for use herein include FGF, hepatocyte growth factor, and the like. Non-limiting examples of suitable chemokines for use herein include platelet growth factor 4, interleukin 8, stromal-derived factor 1a, lipid-binding proteins, and the like. Non-limiting examples of pathogen proteins include HIV-1 envelope protein glycoprotein 120, herpes simplex virus envelope proteins glycoproteins B (gB), gC and gD, dengue virus envelop protein, malaria circumsporozoite protein, and the like.

In embodiments, the present disclosure includes a kit for various biomedical purposes such as cell delivery, tissue engineering, shape-filling polymer, and wound healing. In embodiments a kit includes the hydrogel in the present disclosure and desired bioactive agents. Of note, modified heparin could be optionally excluded from hydrogel composition if cues do not incorporate heparin-binding domains. Collagen and gelatin incorporating the same reactive groups similar to hyaluronic acid described herein above (e.g., gelatin —SH) can also employed in this disclosure to render the shear-thinning hydrogel bioactive. As an example, the biochemical cues in this disclosure are selected to promote proliferation, migration, or differentiation of stem cells e.g., neural crest stem cells into myelin-forming cells Schwann cells; or oligodendrocytes or other cell lineages e.g., neurons, depending on the application.

In embodiments, the present disclosure includes a method for using the bioactive supramolecular hydrogel for delivery of bioactive molecules or live cells into subjects in need thereof, such as patients. As an example, the above-described hydrogel is formulated to include biochemical cues and stem cells for delivery in vivo to promote tissue regeneration. In one example, the myelin-forming cells is delivered (oligodendrocytes, neural crest derived Schwann cells) to the brain of shiverer mice (Shi) lacking myelin to promote myelination. However, the hydrogels and embodiments of the present disclosure can be used to deliver any cell type or therapeutic molecules to any tissue or organ including the central or peripheral nervous system, skeletal muscle, skin, tumor sites etc.

In embodiments, the present disclosure includes a method involving the following steps: 1) cells, including but not limited to myelin-forming cells, are mixed with a solution of functionalized hyaluronic acid (HA) e.g., HA-SH; 2) mixing of heparin (Hep) binding biomolecules e.g., FGF with a solution of functionalized Heparin e.g., Hep-SH; 3) mixing solutions (1) and (2) together; 4) mixture (3) is added to the functional SMP e.g., SMP maleimide to induce crosslinking; and 5) injection of the final cell and biomolecule containing hydrogel into lesion sites.

In embodiments, the present disclosure provides a method for development of novel supramolecular hydrogels with adaptable physical bonds, capable of breaking under mechanical stress (shear-thinning behavior) and reforming after removal of stress. The hydrogels in this disclosure hold great advantages and promise in many biomedical applications such as tissue engineering, drug delivery, cancer treatment, wound healing, and cell therapy. The advantages of these hydrogels include: 1) the dynamic host-guest linkages undergo reversible association/dissociation at physiological conditions, making the hydrogels powerful delivery systems due to their injectability; 2) the hydrogels are biocompatible and promising for cell-encapsulation and injection into deep tissues; 3) the dynamic nature of the host-guest complexation bonds reduce cell death during injection due to dissipation of the injection force through rupture of the host-guest linkages, which in turn reduces the disruption of cell membrane; 4) the adaptable host-guest bonds enable modulation of cell microenvironment and allow cell infiltration, attachment, survival, proliferation and/or migration; 5) the SMP employed in hydrogel formation enable an easy tuning of the rheological properties, making the hydrogels excellent scaffolds for 3D encapsulation/culture of various cells; 6) the feasibility of incorporation of broad numbers of cell-directing cues that promote cell function as necessary e.g., attachment, spreading, survival, proliferation, migration and/or differentiation render these hydrogels promising scaffolds for development of 3D tissue constructs for clinical use; 7) the ability to immobilize a specific biochemical signal or a multitude of signals through direct chemical conjugation or immobilization on heparin opens new a venues in cell biology, through probing cell behavior in response to specific cues and provides insight toward understanding the complex cellular pathways; and 8) promising candidates for cell therapies including treatment of central and peripheral nerve injuries through delivery of function myelin-forming cells.

### EXAMPLES

Non-limiting examples of compositions suitable for use herein include those shown in Table 2 below:

TABLE 2

| Composition | Variable Element |
|---|---|
| HA-SH (DS 16%) + Dextrin- | 4-arm PEG-adamantane |
| Maleimide + multi-arm | 6-arm PEG-adamantane |
| PEG-adamantane | 8-arm PEG-adamantane |
| HA-SH (DS 16%) + Dextrin- | PEG-adamantane (10 kDa) |
| Maleimide + 8-arm | PEG-adamantane (20 kDa) |
| PEG-adamantane | PEG-adamantane (30 kDa) |
| | PEG-adamantane (40 kDa) |
| HA-SH (DS 16%) + Dextrin- | HA-SH (DS 10%) |
| Maleimide + 8-arm | HA-SH (DS 15%) |
| PEG-adamantane | HA-SH (DS 25%) |
| | HA-SH (DS 35%) |
| | HA-SH (DS 75%) |
| HA-Maleimide (DS 16%) + Dextrin- | 4-arm PEG-adamantane |
| SH + multi-arm PEG-adamantane | 6-arm PEG-adamantane |
| | 8-arm PEG-adamantane |
| HA-Maleimide (DS 16%) + Dextrin- | PEG-adamantane (10 kDa) |
| SH + multi-arm PEG-adamantane | PEG-adamantane (20 kDa) |
| | PEG-adamantane (30 kDa) |
| | PEG-adamantane (40 kDa) |
| HA-Maleimide (DS 16%) + Dextrin- | HA-Maleimide (DS 10%) |
| SH + multi-arm PEG-adamantane | HA- Maleimide (DS 15%) |
| | HA-Maleimide (DS 25%) |
| | HA-Maleimide (DS 35%) |
| | HA-Maleimide (DS 75%) |
| HA-SH (DS 16%) + Dextrin-acrylate + | 4-arm PEG-adamantane |
| multi-arm PEG-adamantane | 6-arm PEG-adamantane |
| | 8-arm PEG-adamantane |
| HA-SH (DS 16%) + Dextrin-acrylate + | PEG-adamantane (10 kDa) |
| multi-arm PEG-adamantane | PEG-adamantane (20 kDa) |
| | PEG-adamantane (30 kDa) |
| | PEG-adamantane (40 kDa) |
| HA-SH (DS 16%) + Dextrin-acrylate + | HA-SH (DS 10%) |
| multi-arm PEG-adamantane | HA-SH (DS 15%) |
| | HA-SH (DS 25%) |
| | HA-SH (DS 35%) |
| | HA-SH (DS 75%) |
| HA-SH (DS 16%) + Dextrin- | 4-arm PEG-adamantane |
| Norbornene + multi-arm PEG- | 6-arm PEG-adamantane |
| adamantane + Irgacure | 8-arm PEG-adamantane |
| HA-SH (DS 16%) + Dextrin- | PEG-adamantane (10 kDa) |
| Norbornene + multi-arm PEG- | PEG-adamantane (20 kDa) |
| adamantane + Irgacure | PEG-adamantane (30 kDa) |
| | PEG-adamantane (40 kDa) |
| HA-SH (DS 16%) + Dextrin- | HA-SH (DS 10%) |
| Norbornene + multi-arm PEG- | HA-SH (DS 15%) |
| adamantane + Irgacure | HA-SH (DS 25%) |
| | HA-SH (DS 35%) |
| | HA-SH (DS 75%) |
| HA-azide (DS 16%) + Dextrin-strained | 4-arm PEG-adamantane |
| alkyne + multi-arm PEG-adamantane | 6-arm PEG-adamantane |
| | 8-arm PEG-adamantane |
| HA-azide (DS 16%) + Dextrin-strained | PEG-adamantane (10 kDa) |
| alkyne + multi-arm PEG-adamantane | PEG-adamantane (20 kDa) |
| | PEG-adamantane (30 kDa) |
| | PEG-adamantane (40 kDa) |
| HA-azide (DS 16%) + Dextrin-strained | HA-SH (DS 10%) |
| alkyne + multi-arm PEG-adamantane | HA-azide (DS 15%) |
| | HA-azide (DS 25%) |
| | HA-azide (DS 35%) |
| | HA-azide (DS 75%) |
| HA-strained alkyne (DS 16%) + | 4-arm PEG-adamantane |
| Dextrin-azide + multi-arm PEG- | 6-arm PEG-adamantane |
| adamantane | 8-arm PEG-adamantane |
| HA-strained alkyne (DS 16%) + | PEG-adamantane (10 kDa) |
| Dextrin-azide + multi-arm PEG- | PEG-adamantane (20 kDa) |
| adamantane | PEG-adamantane (30 kDa) |
| | PEG-adamantane (40 kDa) |
| HA-strained alkyne (DS 16%) + | HA-strained alkyne (DS 10%) |
| Dextrin-azide + multi-arm PEG- | HA-strained alkyne (DS 15%) |
| adamantane | HA-strained alkyne (DS 25%) |
| | HA-strained alkyne (DS 35%) |
| | HA-strained alkyne (DS 75%) |
| HA-SH (DS 16%) + Hep-SH + Dextrin- | 4-arm PEG-adamantane |
| Maleimide + multi-arm PEG- | 6-arm PEG-adamantane |
| adamantane | 8-arm PEG-adamantane |
| HA-SH (DS 16%) + Hep-SH + | PEG-adamantane (10 kDa) |
| Dextrin-Maleimide + 8-arm PEG- | PEG-adamantane (20 kDa) |
| adamantane | PEG-adamantane (30 kDa) |
| | PEG-adamantane (40 kDa) |
| HA-SH (DS 16%) + Hep-SH + Dextrin- | HA-SH (DS 10%) |
| Maleimide + 8-arm PEG-adamantane | HA-SH (DS 15%) |
| | HA-SH (DS 25%) |
| | HA-SH (DS 35%) |
| | HA-SH (DS 75%) |
| HA-Maleimide (DS 16%) + Hep- | 4-arm PEG-adamantane |
| Maleimide + Dextrin-SH + multi-arm | 6-arm PEG-adamantane |
| PEG-adamantane | 8-arm PEG-adamantane |
| HA-Maleimide (DS 16%) + Hep- | PEG-adamantane (10 kDa) |
| Maleimide + Dextrin-SH + multi-arm | PEG-adamantane (20 kDa) |
| PEG-adamantane | PEG-adamantane (30 kDa) |
| | PEG-adamantane (40 kDa) |
| HA-Maleimide (DS 16%) + Hep- | HA-Maleimide (DS 10%) |
| Maleimide + Dextrin-SH + multi-arm | HA- Maleimide (DS 15%) |
| PEG-adamantane | HA-Maleimide (DS 25%) |
| | HA-Maleimide (DS 35%) |
| | HA-Maleimide (DS 75%) |
| HA-SH (DS 16%) + Hep-SH + Dextrin- | 4-arm PEG-adamantane |
| acrylate + multi-arm PEG-adamantane | 6-arm PEG-adamantane |
| | 8-arm PEG-adamantane |
| HA-SH (DS 16%) + Hep-SH + Dextrin- | PEG-adamantane (10 kDa) |
| acrylate + multi-arm PEG-adamantane | PEG-adamantane (20 kDa) |
| | PEG-adamantane (30 kDa) |
| | PEG-adamantane (40 kDa) |
| HA-SH (DS 16%) + Hep-SH + Dextrin- | HA-SH (DS 10%) |
| acrylate + multi-arm PEG-adamantane | HA-SH (DS 15%) |
| | HA-SH (DS 25%) |
| | HA-SH (DS 35%) |
| | HA-SH (DS 75%) |
| HA-SH (DS 16%) + Hep-SH + Dextrin- | 4-arm PEG-adamantane |
| Norbornene + multi-arm PEG- | 6-arm PEG-adamantane |
| adamantane + Irgacure | 8-arm PEG-adamantane |
| HA-SH (DS 16%) + Hep-SH + Dextrin- | PEG-adamantane (10 kDa) |
| Norbornene + multi-arm PEG- | PEG-adamantane (20 kDa) |
| adamantane + Irgacure | PEG-adamantane (30 kDa) |
| | PEG-adamantane (40 kDa) |
| HA-SH (DS 16%) + Hep-SH + Dextrin- | HA-SH (DS 10%) |
| Norbornene + multi-arm PEG- | HA-SH (DS 15%) |
| adamantane + Irgacure | HA-SH (DS 25%) |
| | HA-SH (DS 35%) |
| | HA-SH (DS 75%) |
| HA-azide (DS 16%) + Hep-azide + | 4-arm PEG-adamantane |
| Dextrin-strained alkyne + multi-arm | 6-arm PEG-adamantane |
| PEG-adamantane | 8-arm PEG-adamantane |
| HA-azide (DS 16%) + Hep-azide + | PEG-adamantane (10 kDa) |
| Dextrin-strained alkyne + multi-arm | PEG-adamantane (20 kDa) |
| PEG-adamantane | PEG-adamantane (30 kDa) |
| | PEG-adamantane (40 kDa) |
| HA-azide (DS 16%) + Hep-azide + | HA-SH (DS 10%) |
| Dextrin-strained alkyne + multi-arm | HA-azide (DS 15%) |
| PEG-adamantane | HA-azide (DS 25%) |
| | HA-azide (DS 35%) |
| | HA-azide (DS 75%) |
| HA-strained alkyne (DS 16%) + Hep- | 4-arm PEG-adamantane |
| strained alkyne + Dextrin-azide + | 6-arm PEG-adamantane |
| multi-arm PEG-adamantane | 8-arm PEG-adamantane |
| HA-strained alkyne (DS 16%) + Hep- | PEG-adamantane (10 kDa) |
| strained alkyne + Dextrin-azide + | PEG-adamantane (20 kDa) |
| multi-arm PEG-adamantane | PEG-adamantane (30 kDa) |
| | PEG-adamantane (40 kDa) |
| HA-strained alkyne (DS 16%) + Hep- | HA-strained alkyne (DS 10%) |
| strained alkyne + Dextrin-azide + | HA-strained alkyne (DS 15%) |
| multi-arm PEG-adamantane | HA-strained alkyne (DS 25%) |
| | HA-strained alkyne (DS 35%) |
| | HA-strained alkyne (DS 75%) |

### Process Sequence

1—Biochemical signals such as growth factors are optionally added to the shear-thinning hydrogel.
2—Blocking agents such as 2-hydroxyethyl maleimide is optionally added to block the excess thiols, if exist.
3—Heparin is optionally added to the shear-thinning hydrogel for immobilization of bioactive molecules, if needed.
4—Each claim can result in many different hydrogels by varying the wt % of HA and PEG-adamantane. For example:

Table 1 above depicts non-limiting examples of the compositions of supramolecular shear-thinning hydrogel developed by mixing HA-thiol (degree of substitution 15%) with functional supramolecular polymer (SMP, 8-arm, Mwt 40 kDa).
5—When Irgacure is used in the system, the hydrogel formation could be triggered by high wavelength UV-light.
6—The green cells represent the shear-thinning hydrogels that contain heparin.

It is to be understood that while the invention has been described in conjunction with the specific embodiments thereof, that the foregoing description and the examples are intended to illustrate and not limit the scope of the invention. It will be understood by those skilled in the art that various changes may be made and equivalents may be substituted without departing from the scope of the invention, and further that other aspects, advantages and modifications will be apparent to those skilled in the art to which the invention pertains. In addition to the embodiments described herein, the present invention contemplates and claims those inventions resulting from the combination of features of the invention cited herein and those of the cited prior art references which complement the features of the present invention. Similarly, it will be appreciated that any described material, feature, or article may be used in combination with any other material, feature, or article, and such combinations are considered within the scope of this invention.

The disclosures of each patent, patent application, and publication cited or described in this document are hereby incorporated herein by reference, each in its entirety, for all purposes.

1. An injectable hydrogel, comprising:
a water-soluble polymer comprising a first functional group;
a host molecule comprising a second functional group, wherein the second functional group is characterized as complementary to the first functional group; and
a guest-terminated star polymer, wherein the guest-terminated star polymer comprises a core unit, a plurality of arms extending from the core unit, and a guest molecule disposed at a terminal end of each of the plurality of arms, and wherein the host molecule and the guest-terminated star polymer are linked by one or more bonds that break under mechanical stress and reform after removal of the mechanical stress.

2. The injectable hydrogel of claim 1, wherein the first functional group is —SH, NH₂, azide, ketone, aldehyde, furfuryl, maleimide, strained ring, allyl, acrylate, acrylate, alkyne, hydrazide, ketone, furfuryl, norbornene, and oxyamine, or diene, pyridyl disulfide.

3. The injectable hydrogel of claim 1, wherein the second functional group is characterized as mono-functional and selected from the group consisting of thiol, azide, maleimide, strained ring, allyl, acrylate, acrylate, alkyne, hydrazide, ketone, aldehyde, furfuryl, norbornene, and oxyamine.

4. The injectable hydrogel of claim 1, wherein the guest molecule is selected from the group consisting of: adamantane, ferrocene, isopropyl, azobenzene or their derivatives, any other hydrophobic compounds, and combinations thereof.

5. The injectable hydrogel of claim 1, wherein the host molecule is β-cyclodextrin.

6. The injectable hydrogel of claim 1, wherein the water-soluble polymer is hyaluronic acid.

7. (canceled)

8. The injectable hydrogel of claim 1, wherein the one or more bonds are physical bonds characterized as reversible at normal physiological conditions.

9. The injectable hydrogel of claim 1, wherein the hydrogel flows under a shear stress and stiffens after removal of the shear stress.

10. The injectable hydrogel of claim 1, wherein the wherein the plurality of arms comprise functional terminal ends suitable for attaching the guest molecule to the functional terminal ends.

11-13. (canceled)

14. The injectable hydrogel of claim 1, wherein the host-molecule and guest-star polymer physically link to form a multi-arm supramolecular polymer (SMP)-maleimide, SMP-thiol, SMP-acrylate, SMP-norbornene, SMP-strained ring, SMP-azide, or an n-arm-PEG comprising one or more adamantane groups at one or more arm terminal ends, wherein n=2-6.

15.-16. (canceled)

17. The injectable hydrogel of claim 1, further comprising: one or more immobilized biological cues.

18. The injectable hydrogel of claim 17, wherein the immobilized biological cues comprise one or more short peptides, growth factors disposed within a hydrophilic polymer network.

19. The injectable hydrogel of claim 1, further comprising: one or more pharmaceutically active drugs or nutraceuticals; a population of cells; a peptide or peptide derivative; one or more types of nanoparticles or quantum dots; one or more fluorescent or phosphorescent materials; one or more magnetic materials; or a combination thereof.

20. (canceled)

21. The injectable hydrogel of claim 1, wherein a cross-linking reaction provides a physically cross-linked hydrogel having a mechanical stability that is higher than the mechanical stability of the hydrogel before physical cross-linking.

22. The injectable hydrogel of claim 1, wherein the guest-terminated star polymer is an 8-arm-PEG adamantane.

23. (canceled)

24. The injectable hydrogel of claim 1, wherein the guest-terminated polymer is guest-terminated hyperbranched G2-PEG20k-OH, hyperbranched G3-PEG20k-OH, hyperbranched G4-PEG20k-OH, hyperbranched G2-PEG10k-OH, hyperbranched G3-PEG10k-OH, hyperbranched G4-PEG10k-OH, hyperbranched G2-PEG6k-OH, or hyperbranched G3-PEG6k-OH, hyperbranched G4-PEG6k-OH.

25. A shear-thinning supramolecular hydrogel, comprising:
a water-soluble polymer comprising a first functional group;
a host molecule comprising a second functional group, wherein the second functional group is characterized as complementary to the first functional group; and
a guest-terminated star polymer, wherein the guest-terminated star polymer comprises a core unit, a plurality of arms extending from the core unit, and a guest molecule disposed at a terminal end of each of the plurality of arms, and wherein the host molecule and the guest-terminated star polymer are linked by one or more bonds that break under mechanical stress and reform after removal of the mechanical stress.

26. The shear-thinning supramolecular hydrogel of claim 25, wherein the first functional group is —SH, NH₂, azide, ketone, aldehyde, furfuryl, maleimide, strained ring, allyl, acrylate, acrylate, alkyne, hydrazide, ketone, furfuryl, norbornene, and oxyamine, or diene, pyridyl disulfide.

27. The shear-thinning supramolecular hydrogel of claim 25, wherein the second functional group is characterized as mono-functional and selected from the group consisting of thiol, azide, maleimide, strained ring, allyl, acrylate, acrylate, alkyne, hydrazide, ketone, aldehyde, furfuryl, norbornene, and oxyamine.

28. The shear-thinning supramolecular hydrogel of claim 25, wherein the guest molecule is selected from the group consisting of: adamantane, ferrocene, isopropyl, azobenzene or their derivatives, any other hydrophobic compounds, and combinations thereof.

29-61. (canceled)

62. A method of making a hydrogel, comprising: contacting (1) a water-soluble polymer comprising a first functional group, (2) a host molecule comprising a second functional group, wherein the second functional group is characterized as complementary to the first functional group, and (3) a guest-terminated star polymer, wherein the guest-terminated star polymer comprises a core unit, a plurality of arms extending from the core unit, and a guest molecule disposed at a terminal end of each of the plurality of arms, and wherein the host molecule and the guest-terminated star polymer are linked by one or more bonds that break under mechanical stress and reform after removal of the mechanical stress to form a mixture, wherein the contacting is performed under conditions suitable for forming a hydrophilic polymer network from the mixture.

63. The method of making a hydrogel of claim 62, the water-soluble polymer comprises one or more of hyaluronic acid, gelatin, alginate, agarose, chitosan, dextran, collagen, fibronectin, or combinations thereof.

64. The method of making a hydrogel of claim 62, wherein the guest-terminated polymer is one or more of guest-terminated hyperbranched G2-PEG20k-OH, hyperbranched G3-PEG20k-OH, hyperbranched G4-PEG20k-OH, hyperbranched G2-PEG10k-OH, hyperbranched G3-PEG10k-OH, hyperbranched G4-PEG10k-OH, hyperbranched G2-PEG6k-OH, or hyperbranched G3-PEG6k-OH, hyperbranched G4-PEG6k-OH, or combinations thereof.

65. The method of making a hydrogel of claim 62, further comprising contacting the mixture with one or more immobilized biological cues.

66. The method of making a hydrogel of claim 65, wherein the immobilized biological cues comprise one or more short peptides, growth factors disposed within a hydrophilic polymer network.

67. The method of making a hydrogel of claim 62, wherein the mixture further comprises one or more pharmaceutically active drugs or nutraceuticals; a population of cells; a peptide or peptide derivative; one or more types of nanoparticles or quantum dots; one or more fluorescent or phosphorescent materials; one or more magnetic materials; or a combination thereof.

68. The method of making a hydrogel of claim 62, further comprising adding a protein comprising a heparin binding domain and a cell binding domain to the mixture, wherein the heparin-binding domain enables binding to functionalized heparin, and wherein the functionalized heparin comprises a functional thiol group.

69. The method of making a hydrogel of claim 68, wherein the protein is a heparin binding growth factor or a fusion-heparin-binding protein.

70. (canceled)

71. The method of making a hydrogel of claim 69, wherein the fusion-heparin-binding protein comprises HBD-(REDV)n, HBD-(RGDS)n, where n=number of peptide repeats (typically from 3-5).

72. The method of making a hydrogel of claim 62, wherein the hydrogel is further contacted with cells and/or bioactive molecules, or short peptides with reactive groups (REDV-SH, RGD-SH, IKVAV-SH) for chemical attachment within the hydrogel.

73-85. (canceled)
